(19) United States
(12) Patent Application Publication
(10) Pub. No.: US 2022/0370380 A1
(43) Pub. Date: Nov. 24, 2022

(54) EXERCISE-INDUCED HEMOLYSIS SUPPRESSANT AND COMPOSITION FOR SUPPRESSING/IMPROVING EXERCISE-INDUCED HEMOLYTIC ANEMIA

(71) Applicants: AstaReal Co., Ltd., Toyama (JP); Tokai University Educational System, Tokyo (JP)

(72) Inventors: Nobuko Hongo, Toyama (JP); Rika Hirashima, Toyama (JP); Kumi Tominaga, Toyama (JP); Yasuhiro Nishizaki, Tokyo (JP)

(73) Assignees: AstaReal Co., Ltd., Toyama (JP); Tokai University Educational System, Tokyo (JP)

(21) Appl. No.: 17/772,032

(22) PCT Filed: Oct. 29, 2020
(86) PCT No.: PCT/JP2020/040730

§ 371 Date: Apr. 26, 2022

(30) Foreign Application Priority Data
Oct. 30, 2019 (JP) 2019-198026

(51) Int. Cl. A61K 31/122; A61K 9/00; A61P 7/06

(52) U.S. Cl. CPC A61K 31/122; A61K 9/0056; A61P 7/06

(57) ABSTRACT

[Problem] To provide a composition or the like that suppresses the physical destruction of red blood cells (hemolysis) due to exercises and behaviors associated with impact such as striking, for example, as when continually hitting the soles of the feet on the ground by continued running, by using astaxanthin which has a long history of use as a food, and to provide a composition or the like for making the number of red blood cells destroyed (hemolysis number) to lower than the number of red blood cells newly created by hematopoiesis (hematopoiesis number), to suppress and/or improve exercise-induced hemolytic anemia caused by the physical destruction of red blood cells (hemolysis).

[Solution] Astaxanthin is used as an active ingredient.

### TECHNICAL FIELD

The present invention relates to an exercise-induced hemolysis suppressant and a composition for suppressing/improving exercise-induced hemolytic anemia, more specifically, a composition for suppressing exercise-induced hemolysis, using astaxanthin as an active ingredient, a composition for suppressing and/or improving exercise-induced hemolytic anemia, using astaxanthin as an active ingredient, a food or drink for suppressing exercise-induced hemolysis, using astaxanthin as an active ingredient, a food or drink for suppressing and/or improving exercise-induced hemolytic anemia, using astaxanthin as an active ingredient, an exercise-induced hemolysis suppressant using astaxanthin as an active ingredient, an exercise-induced hemolytic anemia suppressing and/or improving agent using astaxanthin as an active ingredient, use of astaxanthin for suppressing exercise-induced hemolysis, use of astaxanthin for suppressing and/or improving exercise-induced hemolytic anemia, and a method for suppressing and/or improving exercise-induced hemolytic anemia by suppressing exercise-induced hemolysis using astaxanthin.

### BACKGROUND ART

Anemia is a condition in which the hemoglobin concentration in the blood unit volume decreases below the normal range due to hemolysis, hemorrhage, blood dilution, hematopoietic failure, or the like. During anemia, symptoms following the decrease of oxygen-carrying capacity such as a sense of malaise and loss of concentration occur, and circulatory or respiratory symptoms such as palpitation and breathlessness occur as a mechanism to compensate for anemia. FIG. 1 shows the classification of the causes of anemia.

Conventionally, anemia peculiar to athletes is known, and it is called sports anemia or exercise-induced anemia, as shown in FIG. 1. Then, the sports anemia (exercise-induced anemia) is classified into four categories: iron-deficiency, hemolytic, hemorrhagic, and dilutional (Non Patent Literature 1). The exercise-induced iron-deficiency anemia occurs most frequently among exercise-induced anemia and is anemia that develops due to low iron supply or high iron excretion, resulting in iron deficiency and decreased hemoglobin synthesis in erythroblasts, like normal anemia. As countermeasures, measures such as taking iron-rich ingredients (supply measure), taking iron-containing supplements or drugs (supply measure), supplying iron directly into blood by intravenous injection (supply measure), and refraining from exercising (excretion measure) are conventionally taken. Further, the exercise-induced hemolytic anemia (exercise-induced hemolytic anemia) is anemia that is caused by physical destruction of erythrocytes (red blood cells; hemolysis) due to exercises and behaviors associated with impact such as striking, for example, as when continually hitting the soles of the feet on the ground by continued running and develops when the number of erythrocytes destroyed (number of hemolysis) exceeds the number of erythrocytes newly created (produced) by hematopoiesis (number of hematopoiesis). As countermeasures, measures such as refraining from exercising, laying insoles on shoes, and wearing thick-soled sneakers are conventionally taken. Though anemia, for example, due to minute bleeding in the digestive tract caused by intense exercise has been reported, exercise-induced hemorrhagic anemia rarely develops (Non Patent Literature 1). However, the exercise-induced hemorrhagic anemia is accompanied by severe symptoms and requires treatment by a doctor. The exercise-induced dilutional anemia, also called apparent anemia, is anemia that develops due to an increase in circulating plasma volume and does not require treatment.

That is, the exercise-induced anemia (sports anemia) is generally anemia caused by iron deficiency and hemolysis, and most athletes who have developed the anemia are currently relying on iron supply such as taking iron-rich ingredients, taking iron-containing supplements or drugs, or supplying iron directly into blood by intravenous injection. Nowadays, there are many cases of, in addition to cases of excessively taking iron without knowing that excessive iron intake may have a serious adverse effect on the human body, directly administering iron by injection despite having no iron deficiency, taking iron for increasing the performance despite having no iron deficiency, and taking iron as a preventive measure against anemia without careful consideration, and health hazards caused by them have become problematic, so that the Japan Association of Athletics Federations (JAAF) has issued a warning (http://www.med.or.jp/sportsdoctor/wp-content/uploads/2019/03/tetuzai_rikuren.pdf). In particular, the current situation is such that there could be found no foods or drinks, or no agents that are effective for suppressing or improving the exercise-induced hemolytic anemia, whereas there is often no need to rely on iron supply for suppressing or improving the exercise-induced hemolytic anemia.

Meanwhile, astaxanthin (also astaxanthine, 3,3′-dihydroxy-β,β-carotene-4,4′-dione) is a red-orange pigment substance with long history of use as a food that is a kind of carotenoids like β-carotene of carrots or lycopene of tomatoes and classified as xanthophylls, and it has been used as a pigment in food additives or a color enhancer for farmed fish over the years. Also, it has been used for pharmaceutical products, health foods such as supplements, basic cosmetics, or the like, since it has been found to have excellent antioxidant activity that is 1000 times higher than that of vitamin E. Since then, various actions and effects of astaxanthin have been found, and its application is steadily expanding.

Though not related to the exercise-induced hemolytic anemia, it has been reported that astaxanthin is used for autoimmune hemolytic anemia (hemolytic anemia due to immunoinflammatory disorders) (Patent Literature 1). Further, it has been reported that hemolysis due to dilution is suppressed by the antioxidant activity of astaxanthin exerting an effect of protecting the erythrocyte membrane from oxidative damage (Patent Literature 2).

### CITATION LIST

### Patent Literature

Patent Literature 1: Japanese Translation of PCT International Application Publication No. 2007-538083
Patent Literature 2: Japanese Patent Laid-Open No. 2002-226368

### Non Patent Literature

Non Patent Literature 1: Motoaki HIRASAWA “Long-distance running and exercise-induced anemia in high school students” Reitaku University journal, Volume 96, p 90, July 2013

### SUMMARY OF INVENTION

### Technical Problem

However, the effect of suppressing hemolysis due to oxidative damage (oxidative damage to erythrocytes (red blood cells)) is different from the effect of suppressing physical destruction of erythrocytes (hemolysis) due to exercises and behaviors associated with impact such as striking, for example, as when continually hitting the soles of the feet on the ground by continued running, as revealed also in Examples of this description. Meanwhile, autoimmune hemolysis (hemolysis due to immunoinflammatory disorders) is different from hemolysis due to exercises and behaviors associated with impact such as striking (physical destruction of erythrocytes due to exercises and behaviors associated with impact such as striking) since it corresponds to the “hemolysis due to autoantibody abnormalities” shown in FIG. 1. Accordingly, it can be said that the effect of suppressing autoimmune hemolytic anemia (anemia caused by hemolysis due to immunoinflammatory disorders) is also different from the effect of suppressing anemia caused by hemolysis due to exercises and behaviors associated with impact such as striking (anemia caused by physical destruction of erythrocytes due to exercises and behaviors associated with impact such as striking).

The present invention has been devised in order to solve the problems such as a problem of excessively taking iron without knowing that excessive iron intake may have a serious adverse effect on the human body, despite having exercise-induced hemolytic anemia, as mentioned above, a problem of directly administering iron by injection despite having no iron deficiency, a problem of taking iron for increasing the performance despite having no iron deficiency, and a problem of taking iron as a preventive measure against anemia without careful consideration. It is an object of the present invention to provide a composition or the like that suppresses the physical destruction of erythrocytes (hemolysis) due to exercises and behaviors associated with impact such as striking, for example, as when continually hitting the soles of the feet on the ground by continued running, by using astaxanthin which has a long history of use as a food, and to provide a composition or the like for making the number of erythrocytes (red blood cells) destroyed (hemolysis number) to lower than the number of erythrocytes newly created (produced) by hematopoiesis (hematopoiesis number), to suppress and/or improve exercise-induced hemolytic anemia caused by the physical destruction of erythrocytes (red blood cells; hemolysis).

### Solution to Problem

As a result of dedicated studies, the inventors have found that astaxanthin (including free forms and derivatives) suppresses the physical destruction of erythrocytes (red blood cells) due to exercises and behaviors associated with impact such as striking, for example, continuously hitting the soles of the feet on the ground by continuous running (hemolysis), as a result of which, the number of erythrocytes destroyed (hemolysis number) is reduced to lower than the number of erythrocytes newly created (produced) by hematopoiesis (hematopoiesis number), so that so-called exercise-induced hemolytic anemia is suppressed and/or improved, thereby accomplished the invention as follows.

(1) A composition for suppressing exercise-induced hemolysis, using astaxanthin as an active ingredient.
(2) A composition for suppressing and/or improving exercise-induced hemolytic anemia, using astaxanthin as an active ingredient.
(3) The composition according to (1) or (2), wherein the composition is a food or drink composition or a pharmaceutical composition.
(4) The composition according to any one of (1) to (3), wherein the exercise-induced hemolysis excludes hemolysis due to oxidative damage.
(5) The composition according to any one of (1) to (4), wherein the astaxanthin is derived from Haematococcus algae extract.
(6) A food or drink for suppressing exercise-induced hemolysis, using astaxanthin as an active ingredient.
(7) A food or drink for suppressing and/or improving exercise-induced hemolytic anemia, using astaxanthin as an active ingredient.
(8) The food or drink according to (6) or (7), wherein the exercise-induced hemolysis excludes hemolysis due to oxidative damage.
(9) The food or drink according to any one of (6) to (8), wherein the astaxanthin is derived from Haematococcus algae extract.
(10) An exercise-induced hemolysis suppressant using astaxanthin as an active ingredient.
(11) An exercise-induced hemolytic anemia suppressing and/or improving agent using astaxanthin as an active ingredient.
(12) The agent according to (10) or (11), wherein the exercise-induced hemolysis excludes hemolysis due to oxidative damage.
(13) The agent according to any one of (10) to (12), being free from iron.
(14) The agent according to any one of (10) to (13), wherein the astaxanthin is derived from Haematococcus algae extract.
(15) Use of astaxanthin for suppressing exercise-induced hemolysis.
(16) Use of astaxanthin for suppressing and/or improving exercise-induced hemolytic anemia.
(17) The use according to (15) or (16), wherein the exercise-induced hemolysis excludes hemolysis due to oxidative damage.
(18) The use according to any one of (15) to (17), being use as a food or drink or a drug.
(19) The use according to any one of (15) to (18), wherein iron is not used together.
(20) The use according to any one of (15) to (19), wherein the astaxanthin is derived from Haematococcus algae extract.
(21) A method for suppressing and/or improving exercise-induced hemolytic anemia by suppressing exercise-induced hemolysis using astaxanthin.
(22) The method according to (21), wherein the exercise-induced hemolysis excludes hemolysis due to oxidative damage.
(23) The method according to (21) or (22), wherein the astaxanthin is used as a food or drink or a drug.
(24) The method according to any one of (21) to (23), wherein iron is not used.
(25) The method according to any one of (21) to (24), wherein the astaxanthin is derived from Haematococcus algae extract.

### Advantageous Effects of Invention

According to the present invention, exercise-induced hemolysis, that is, physical destruction of erythrocytes (red blood cells) due to exercises and behaviors associated with impact such as striking, for example, continuously hitting the soles of the feet on the ground by continuous running (hemolysis) can be suppressed, further the number of erythrocytes destroyed (number of hemolysis) can be reduced to lower than the number of erythrocytes newly created (produced) by hematopoiesis (number of hematopoiesis), and exercise-induced hemolytic anemia caused by the physical destruction of erythrocytes (hemolysis) can be suppressed and/or improved. As a result, not only iron supply is not relied on, but also problems such as a problem of excessively taking iron without knowing that excessive iron intake may have a serious adverse effect on the human body despite having exercise-induced hemolytic anemia, a problem of directly administering iron by injection despite having no iron deficiency, a problem of taking iron for increasing the performance despite having no iron deficiency, and a problem of taking iron as a preventive measure against anemia without careful consideration can be overcome.

### BRIEF DESCRIPTION OF DRAWINGS

FIG. 1 is a diagram showing classification of the causes of anemia.

FIG. 2 is a table showing the average number of steps and the average travel distance of the subjects in Examples of this description and ordinary people.

FIG. 3 is a table showing changes in the number of erythrocytes (red blood cells) and the hemoglobin content (amount of blood pigment; hemoglobin concentration) of ordinary people.

FIG. 4 includes graphs showing the rate of change in the number of erythrocytes and its transition during an observation period in each of an “astaxanthin group” and a “control group”, wherein (A) represents the rate of change in the number of erythrocytes in each of the “astaxanthin group” and the “control group” during the observation period, and (B) represents the transition of the rate of change in the number of erythrocytes from the start date of the observation period to the end date of the observation period in each of the “astaxanthin group” and the “control group”.

FIG. 5 includes graphs showing the rate of change in the hemoglobin content (amount of blood pigment; hemoglobin concentration) and its transition during the observation period in each of the “astaxanthin group” and the “control group”, wherein (A) represents the rate of change in the hemoglobin content (hemoglobin concentration) during the observation period in each of the “astaxanthin group” and the “control group”, and (B) represents the transition of the rate of change in the hemoglobin content (hemoglobin concentration) from the start date of the observation period to the end date of the observation period in each of the “astaxanthin group” and the “control group”.

FIG. 6 includes graphs showing the average of measured values of the serum haptoglobin concentration on the start date of the observation period and the end date of the observation period, and the transition of the average of measured values of the serum haptoglobin concentration during the observation period in each of the “astaxanthin group” and the “control group”, wherein (A) is a graph showing the average of measured values of the serum haptoglobin concentration on the end date of the observation period in each of the “astaxanthin group” and the “control group”, and (B) is a graph showing the transition of the average of measured values of the serum haptoglobin concentration from the start date of the observation period to the end date of the observation period in each of the “astaxanthin group” and the “control group”.

FIG. 7 includes a table showing the averages of measured values of the production rates of 8-OHdG and isoprostane at Week 0 (start date of test food intake), Week 4, and Week 8 (last day of test food intake), and graphs showing transitions of the averages of measured values of the production rates of 8-OHdG and isoprostane during the test period in each of the “astaxanthin group” and the “control group”, wherein (A) is a table showing the averages of measured values of the production rates of 8-OHdG and isoprostane at Week 0, Week 4, and Week 8 in each of the “astaxanthin group” and the “control group”, (B) is a graph showing the transition of the averages of measured values of the production rate of 8-OHdG from Week 0 to Week 8, that is, during the test period in each of the “astaxanthin group” and the “control group”, and (C) is a graph showing the transition of the averages of measured values of the production rate of isoprostane from Week 0 to Week 8, that is, during the test period in each of the “astaxanthin group” and the “control group”.

FIG. 8 includes a table showing the averages of measured values of total antioxidant capacity at Week 0 (start date of test food intake), Week 4, and Week 8 (last day of test food intake) in each of the “astaxanthin group” and the “control group”, and a graph showing the transition of the averages of measured values of total antioxidant capacity during the test period, wherein (A) is a table showing the averages of measured values of the total antioxidant capacity at Week 0, Week 4, and Week 8 in each of the “astaxanthin group” and the “control group”, and (B) is a graph showing the transition of the averages of measured values of total antioxidant capacity from Week 0 to Week 8, that is, during the test period in each of the “astaxanthin group” and the “control group”.

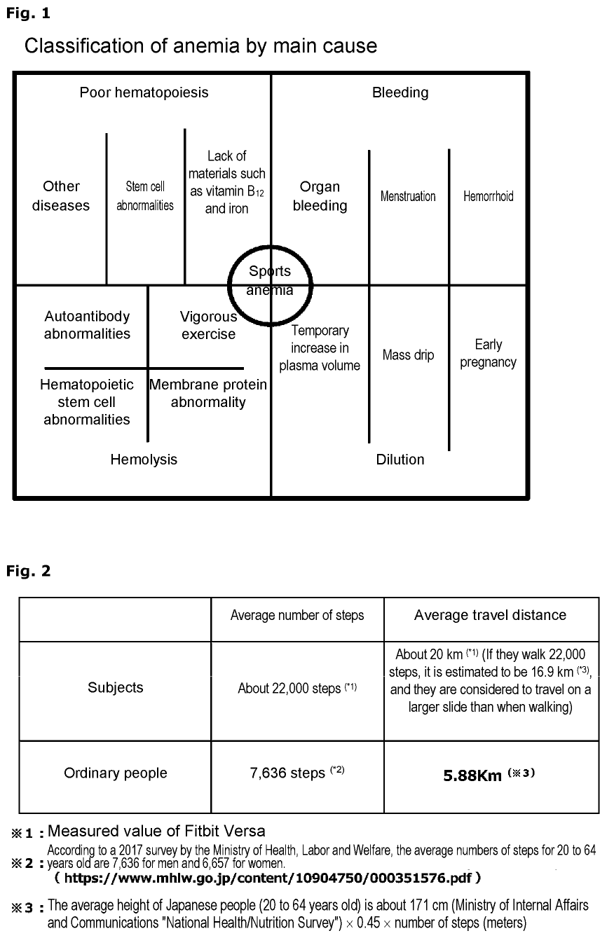

### DESCRIPTION OF EMBODIMENTS

Hereinafter, a composition for suppressing exercise-induced hemolysis, using (containing) astaxanthin as an active ingredient, a composition for suppressing and/or improving exercise-induced hemolytic anemia, using (containing) astaxanthin as an active ingredient, a food or drink for suppressing exercise-induced hemolysis, using (containing) astaxanthin as an active ingredient, a food or drink for suppressing and/or improving exercise-induced hemolytic anemia, using (containing) astaxanthin as an active ingredient, an exercise-induced hemolysis suppressant using (containing) astaxanthin as an active ingredient, an exercise-induced hemolytic anemia suppressing and/or improving agent using (containing) astaxanthin as an active ingredient, use of astaxanthin for suppressing exercise-induced hemolysis, use of astaxanthin for suppressing and/or improving exercise-induced hemolytic anemia, and a method for suppressing and/or improving exercise-induced hemolytic anemia by suppressing exercise-induced hemolysis using astaxanthin, according to the present invention will be described in detail. In the present invention, any of the “composition for suppressing exercise-induced hemolysis”, the “composition for suppressing and/or improving exercise-induced hemolytic anemia”, the “food or drink for suppressing exercise-induced hemolysis”, the “food or drink for suppressing and/or improving exercise-induced hemolytic anemia”, the “exercise-induced hemolysis suppressant”, and the “exercise-induced hemolytic anemia suppressing and/or improving agent” contains astaxanthin as an active ingredient (uses it as an active ingredient). In the present invention, any of the “use for suppressing exercise-induced hemolysis”, the “use for suppressing and/or improving exercise-induced hemolytic anemia”, the “method for suppressing exercise-induced hemolysis”, and the “method for suppressing and/or improving exercise-induced hemolytic anemia” uses astaxanthin as a substance effective for suppressing exercise-induced hemolysis and suppressing and/or improving exercise-induced hemolytic anemia.

Astaxanthin (3,3′-dihydroxy-β,β-carotene-4,4′-dione) is a kind of carotenoids. Carotenoids refer to a group of compounds that are a kind of terpenoids, are yellow to red pigments (carotenoid pigments), and are a general term for aliphatic or alicyclic polyenes containing many conjugated double bonds.

Further, astaxanthin is a red pigment with long history of use as a food that is widely distributed in nature, particularly, in the ocean, for example in crustaceans such as shrimps and crabs, fishes such as salmons and porgies, algae such as green algae Hematococcus, and yeasts such as red yeast Phaffia. Astaxanthin has not only a strong antioxidant activity about 1,000 times that of vitamin E and about 40 times that of β-carotene, but also biological activities such as antioxidant effect, anti-inflammatory effect, skin antiaging effect, and whitening effect, and is known as a pigment ranging from yellow to red. Astaxanthin has three isomers, 3S-3′S form, 3S-3′R form (meso form), and 3R-3′R form, depending on the configuration of the hydroxyl group at the 3(3′)-position of the ring structure present at each of both ends of the molecule. In addition, there are cis and trans geometric isomers of the conjugated double bond in the center of the molecule. For example, there are all-trans, 9-cis form, and 13-cis form. Further, the hydroxyl group at the 3(3′)-position can form an ester form with a fatty acid.

Further, astaxanthin is known to be a highly safe compound with no mutagenicity observed and is widely used as food additives (Jiro TAKAHASHI, et al.: Toxicological Studies of Astaxanthin from Haematococcus pluvialis-Ames test, Oral Single Dose and 90-days Subchronic Toxicity Studies in Rats-Journal of Clinical Therapeutics & Medicines, 20: 867-881, 2004).

In the present invention, the “astaxanthin” includes free forms and/or derivatives such as an ester of astaxanthin, in addition to astaxanthin itself. Further, the ester of astaxanthin includes monoester forms and/or diester forms. For example, the astaxanthin obtained from Haematococcus pluvialis is known to have a 3S-3′S form and contain a large amount of monoester forms in which a fatty acid (one fatty acid) is bound (Renstrom, B. et. al., Fatty acids of some esterified carotenols, Comp. Biochem. Physiol. B, Comp. Biochem., 1981, 69, p. 625-627), and the astaxanthin obtained from krill is known to contain a large amount of diester forms in which two fatty acids are bound (Yamaguchi, K. et. al., The composition of carotenoid pigments in the Antarctic krill Euphausia superba, Bull. Jap. Sos. Sci. Fish., 1983, 49, p. 1411-1415). The “astaxanthin” of the present invention also includes such astaxanthins.

Further, the astaxanthin obtained from Phaffia Rhodozyma is known to have a 3R-3′R form (Andrewes, A. G. et. al., (3R,3′R)-Astaxanthin from the yeast Phaffia rhodozyma, Phytochem., 1976, 15, p. 1009-1011), has a structure opposite to the 3S-3'S forms normally found in nature, and is present as a non-ester form, that is, free form, which does not form an ester with a fatty acid (Andrewes, A. G. et. al., Carotenids of Phaffia rhodozyma, a red pigmented fermenting yeast, Phytochem., 1976, 15, p. 1003-1007). The “astaxanthin” of the present invention also includes such an astaxanthin.

Meanwhile, the “astaxanthin” in the present invention includes not only natural astaxanthins, but also synthetic astaxanthins. Examples of the natural astaxanthins can include astaxanthin-containing extracts obtained from algae such as Haematococcus; yeasts such as Phaffia; crustaceans such as shrimps, krill, and crabs; cephalopods such as squids and octopuses; various marine products; plants such as Adonis; bacteria such as Paracoccus sp. N81106, Brevundimonas sp. SD212, and Erythrobacter sp. PC6; actinomycetes such as Gordonia sp. KANMONKAZ-1129; Labyrinthulas such as Schizochytrium sp. KH105; astaxanthin-producing gene recombinant organisms, and astaxanthins appropriately purified from the astaxanthin-containing extracts, preferably astaxanthins derived from microalgae extracts extracted from microalgae such as Haematococcus, more preferably astaxanthins derived from Haematococcus algae extract extracted from Haematococcus algae. Further, examples of the synthetic astaxanthins can include AstaSana (Koninklijke DSM N.V.) and Lucantin Pink® (BASF SE). Further, examples of synthetic astaxanthins obtained by chemically converting other naturally occurring carotenoids can include AstaMarine (PIVEG, INC.).

Examples of the Haematococcus algae from which natural astaxanthins are obtained can include Haematococcus pluvialis, Haematococcus lacustris, Haematococcus capensis, Haematococcus deroebakensis, and Haematococcus zimbabwiensis.

The method for culturing these Haematococcus green algae is preferably a closed-type culture method that is free from contamination/reproduction of foreign microorganisms with less contamination of other foreign substances. Examples thereof can include, in addition to a culture method using a partially open dome-shaped, conical or cylindrical culture apparatus and a culture medium having a gas discharge device movable in the apparatus (International Publication No. 1999/050384), a method of inducing cystization of algae by applying a drought stress to Haematococcus algae and collecting astaxanthin from the culture of the cystized algae (Japanese Patent Laid-Open No. 8-103288), a culture method by putting a light source in a closed-type culture apparatus, followed by irradiation with light from the inside, and a method using a flat plate-shaped culture tank or a tube-shaped culture layer.

Further, the “astaxanthin” in the present invention also includes astaxanthin-containing extracts extracted from the Haematococcus algae, for example, by crushing the cell wall according to the method disclosed in Japanese Patent Laid-Open No. 5-068585 or the like, as required, and adding an organic solvent such as acetone, ether, chloroform, and an alcohol (such as ethanol and methanol) or an extraction solvent/a solvent such as carbon dioxide in a supercritical state, and the products of the astaxanthin-containing extracts appropriately purified, as required. The astaxanthin content in such an astaxanthin-containing extract is preferably 3 to 40% (w/w), more preferably 3 to 12% (w/w), further preferably 5 to 10% (w/w).

Further, the “astaxanthin” in the present invention also includes commercially available astaxanthins. Examples of the commercially available products can include ASTAREALs such as AstaReal Oil 200SS (a fat-soluble extract from Haematococcus algae, containing about 20% of astaxanthin in terms of free forms), AstaReal L10, AstaReal Oil 50F, AstaReal Oil 50FC, AstaReal Oil 5F, AstaReal P2AF, AstaTROL-X, AstaReal Oil 50FC, ASTAREAL powder 20F, water-soluble ASTAREAL liquid, ASTAREAL WS liquid, ASTAREAL 10WS liquid, ASTAREAL ACT, Astavita e, Astavita sports, Astavita Zenshin, Akai megumi, and astamate, Astavita and astamate Series (all are registered trademarks; available from AstaReal, Inc. and Fuji Chemical Industries Co., Ltd.); ASTOTS Series such as ASTOTS-S, ASTOTS-100, ASTOTS-ECS, ASTPTS-2.0PW, and ASTOTS-3.0 MB (all are registered trademarks; available from FUJIFILM Corporation); BioAstin (Registered trademark; available from Cyanotech Corporation); Astazine™ (available from BGG Japan); astaxanthin powder 1.5%, astaxanthin powder 2.5%, astaxanthin oil 5%, and astaxanthin oil 10% (available from Bio Actives Japan Corporation); astaxanthin (available from Oryza Oil & Fat Chemical Co., Ltd.); SunActive AX (Registered trademark; available from Taiyo Kagaku Co., Ltd.); Haematococcus WS30 (available from Yaegaki Biotechnology, Inc.); and AstaMarine (PIVEG, INC.).

It should be noted that AstaReal Oil 200SS, AstaReal Oil 50FC, AstaReal P2AF, AstaTROL-X, AstaReal Oil 50FC, and ASTAREAL powder 20F, available from AstaReal, Inc. and Fuji Chemical Industries Co., Ltd. each have received “Halal certification”, and AstaReal Oil 50FC, AstaReal P2AF, AstaTROL-X, AstaReal Oil 50FC, and ASTAREAL powder 20F, available from AstaReal, Inc. and Fuji Chemical Industries Co., Ltd. each have received “kosher” certification. Further, AstaReal L10 has received Non-GMO (non-genetically modified organism certification).

Meanwhile, the “exercise-induced hemolysis” refers to physical destruction of erythrocytes (red blood cells; hemolysis) due to exercises and behaviors associated with impact such as striking, for example, as when continually hitting the soles of the feet on the ground by continued running, as described above, and the “exercise-induced hemolytic anemia” is anemia that is caused by the “exercise-induced hemolysis” and develops when the number of erythrocytes destroyed (number of hemolysis) exceeds the number of erythrocytes newly created (produced) by hematopoiesis (number of hematopoiesis), as described above. Examples of the exercises that may cause the “exercise-induced hemolysis” and the “exercise-induced hemolytic anemia” to develop can include, in addition to exercises in which the soles are continuously hit on the ground, including long-distance running such as marathon, triathlon, and racewalking, and ball games such as volleyball, basketball, soccer, rugby, American football, and tennis, exercises and behaviors in which the body can be continuously hit or impacted, including martial arts such as boxing, kickboxing, karate, aikido, jujutsu (Japanese art of self-defence), judo, taekwondo, wrestling, and Kendo (Japanese fencing), and behaviors to continuously handle rolling machines, compaction machines, and the like, such as plate compactors and tamping rammers.

That is, the “composition for suppressing exercise-induced hemolysis, using (containing) astaxanthin as an active ingredient”, the “food or drink for suppressing exercise-induced hemolysis, using (containing) astaxanthin as an active ingredient”, and the “exercise-induced hemolysis suppressant using (containing) astaxanthin as an active ingredient” in the present invention refer to a composition, a food or drink, or an agent that suppresses physical destruction of erythrocytes (hemolysis) by exercises in which the soles are continuously hit on the ground or exercises and behaviors in which the body can be continuously hit or impacted. In the present invention, the “composition for suppressing and/or improving exercise-induced hemolytic anemia, using (containing) astaxanthin as an active ingredient”, the “food or drink for suppressing and/or improving exercise-induced hemolytic anemia, using (containing) astaxanthin as an active ingredient”, and the “exercise-induced hemolytic anemia suppressing and/or improving agent using (containing) astaxanthin as an active ingredient” refer to a composition, a food or drink, or an agent that suppresses and/or improves anemia caused by the physical destruction of erythrocytes (hemolysis), due to the number of erythrocytes (red blood cells) destroyed (number of hemolysis) being reduced as compared with the number of erythrocytes newly created by hematopoiesis (number of hematopoiesis), as a result of suppressing the physical destruction of erythrocytes (hemolysis) caused by exercises in which the soles are continuously hit on the ground or exercises and behaviors in which the body can be continuously hit or impacted. Accordingly, the “composition for suppressing exercise-induced hemolysis, using (containing) astaxanthin as an active ingredient”, the “food or drink for suppressing exercise-induced hemolysis, using (containing) astaxanthin as an active ingredient”, and the “exercise-induced hemolysis suppressant using (containing) astaxanthin as an active ingredient” in the present invention can be the “composition for suppressing and/or improving exercise-induced hemolytic anemia, using (containing) astaxanthin as an active ingredient”, the “food or drink for suppressing and/or improving exercise-induced hemolytic anemia, using (containing) astaxanthin as an active ingredient”, and the “exercise-induced hemolytic anemia suppressing and/or improving agent using (containing) astaxanthin as an active ingredient” in the present invention.

Meanwhile, the “use of astaxanthin for suppressing exercise-induced hemolysis” in the present invention refers to use of astaxanthin for suppressing physical destruction of erythrocytes (hemolysis) caused by exercises in which the soles are continuously hit on the ground or exercises and behaviors in which the body can be continuously hit or impacted. The “use of astaxanthin for suppressing and/or improving exercise-induced hemolytic anemia” and the “method for suppressing and/or improving exercise-induced hemolytic anemia by suppressing exercise-induced hemolysis using astaxanthin” in the present invention refer to use of astaxanthin for suppressing and/or improving anemia caused by the physical destruction of erythrocytes (hemolysis) by suppressing physical destruction of erythrocytes (hemolysis) by exercises in which the soles are continuously hit on the ground or exercises and behaviors in which the body can be continuously hit or impacted and reducing the number of erythrocytes (red blood cells) destroyed (number of hemolysis) to lower than the number of erythrocytes newly created by hematopoiesis (number of hematopoiesis), or use method thereof.

In this description, the term “suppress” may be used interchangeably with the term “prevent” or “inhibit”, and the term “improve” may be used interchangeably with the term “recover” or “treat”.

Here, FIG. 2 shows the average number of steps and the average travel distance of the subjects in Examples of this description and ordinary people. As shown in FIG. 2, the subjects in Examples of this description have a large average number of steps per day and a long average travel distance as compared with ordinary people, and therefore it is understood from FIG. 2 that the subjects in Examples of this description were “running”.

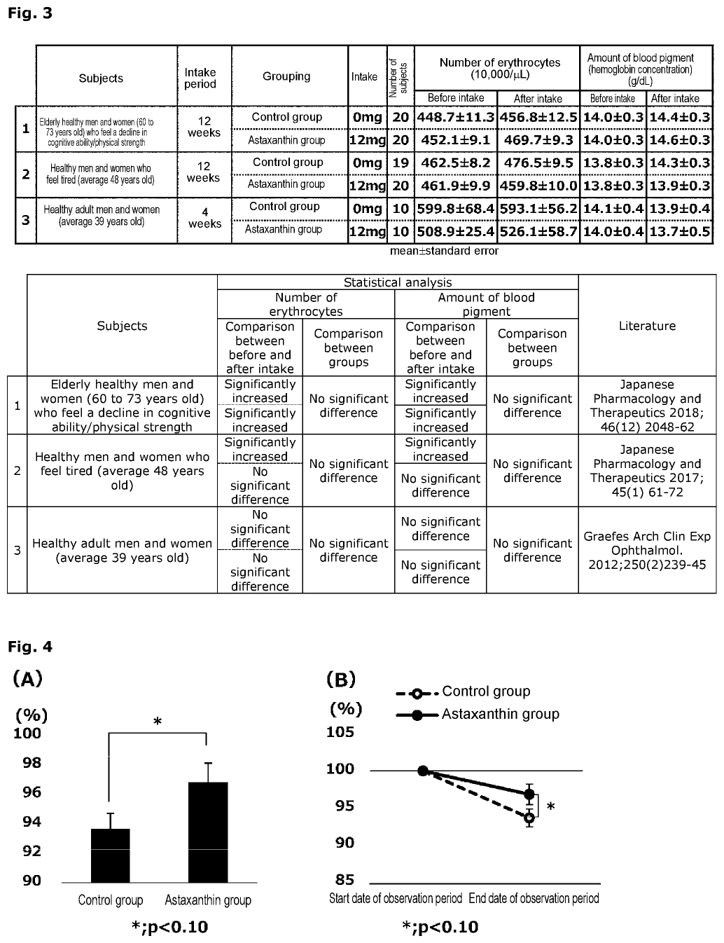

Further, FIG. 3 shows changes in the number of erythrocytes (red blood cells) and the hemoglobin content (amount of blood pigment; hemoglobin concentration) of ordinary people. As shown in FIG. 3, there was no significant difference in the number of erythrocytes and in hemoglobin content (hemoglobin concentration) in the “astaxanthin group” with the test food containing astaxanthin taken and the “control group” with the test food free from astaxanthin taken, between before and after taking the test food. Accordingly, it is understood that, since physical destruction of erythrocytes (hemolysis) does not occur in ordinary people who do not exercise vigorously, no significant decrease in the number of erythrocytes and hemoglobin content (hemoglobin concentration) was observed, and no suppression of the decrease in the number of erythrocytes and in hemoglobin content (hemoglobin concentration) by taking astaxanthin was observed.

Then, the “exercise-induced hemolysis” in the present invention excludes hemolysis due to oxidative damage. This was clarified in Examples of this description, as described above. Whether or not hemolysis due to oxidative damage is suppressed can be confirmed and determined by a technique appropriately selectable by those skilled in the art. Examples of the technique can include a technique of measuring the production rate of 8-OHdG or isoprostane contained in the urine of subjects. The reasons are as follows. Urinary 8-OHdG is a biomarker reflecting the oxidative damage to DNA due to oxidative stress such as active oxygen. The magnitude of oxidative stress and the level of oxidative damage to DNA can be evaluated by measuring the production rate of 8-OHdG. Further, urinary isoprostane is a prostaglandin-like compound formed by oxidation of phospholipids by free radicals, and lipid oxidation in vivo can be evaluated by measuring the production rate of isoprostane.

Further, Examples of this description clarified that the effect of “suppressing exercise-induced hemolysis” and the effect of “suppressing exercise-induced hemolytic anemia” in the present invention are not associated with the antioxidant capacity of astaxanthin. Whether or not they are associated with the antioxidant capacity can be confirmed and determined by a technique appropriately selectable by those skilled in the art. Examples of the technique can include a technique of measuring the total antioxidant capacity by measuring the concentrations of water-soluble antioxidants contained in the serum of subjects. This is because a total antioxidant capacity (Serum Total Antioxidant Status) against oxidative stress can be known by detecting the water-soluble antioxidants in the serum.

Then, the “composition for suppressing exercise-induced hemolysis, using (containing) astaxanthin as an active ingredient”, the “composition for suppressing and/or improving exercise-induced hemolytic anemia, using (containing) astaxanthin as an active ingredient”, the “food or drink for suppressing exercise-induced hemolysis, using (containing) astaxanthin as an active ingredient”, the “food or drink for suppressing and/or improving exercise-induced hemolytic anemia, using (containing) astaxanthin as an active ingredient”, the “exercise-induced hemolysis suppressant using (containing) astaxanthin as an active ingredient”, and the “exercise-induced hemolytic anemia suppressing and/or improving agent using (containing) astaxanthin as an active ingredient” in the present invention do not have to contain iron. In the present invention, the “iron” refers to a compound that produces divalent or trivalent iron ions when dissolved in water. Examples of the “iron” can include ferric ammonium citrate, ferrous fumarate, ferric chloride, sodium ferrous citrate, sodium ferrous gluconate, ferrous gluconate, ferrous lactate, iron pyrophosphate, ferrous sulfate, yellow iron oxide, yellow ferric oxide, brown iron oxide, black iron oxide, iron sesquioxide, ferrous fumarate, and ferrous sulfate.

Then, the “composition for suppressing exercise-induced hemolysis, using (containing) astaxanthin as an active ingredient” and the “composition for suppressing and/or improving exercise-induced hemolytic anemia, using (containing) astaxanthin as an active ingredient” in the present invention are preferably a food or drink composition or a pharmaceutical composition. Examples of the food or drink composition can include foods or drinks such as supplements, solid foods, fluid foods, and beverages, which are prepared by a technique appropriately selectable by those skilled in the art. Examples of the pharmaceutical composition can include general formulations including oral agents such as water agents, tablets, capsules, granules, fine grains, powders, chewable agent, suspending agent, emulsions, syrups, and elixirs, and parenteral agents such as injections, suppositories, inhalants, nasal preparations, and transdermal agents, which are prepared by a technique appropriately selectable by those skilled in the art. The oral agents are preferable. Such formulations can be prepared by a conventional method using the “composition for suppressing exercise-induced hemolysis, using (containing) astaxanthin as an active ingredient” and the “composition for suppressing and/or improving exercise-induced hemolytic anemia, using (containing) astaxanthin as an active ingredient” according to the present invention and pharmaceutically acceptable other components such as excipients.

Further, the “composition for suppressing exercise-induced hemolysis, using (containing) astaxanthin as an active ingredient” and the “composition for suppressing and/or improving exercise-induced hemolytic anemia, using (containing) astaxanthin as an active ingredient” in the present invention can be taken as foods for special use such as foods for special dietary uses (food for specified health uses), foods with function claims, foods with nutrient function claims, baby modified milk powder, infant milk powder, lactating woman milk powder, foods with health function claims, foods for patients, milk products, and fermented milk, expecting the effect of suppressing exercise-induced hemolysis or the effect of suppressing and/or improving exercise-induced hemolytic anemia. Further, they can be taken as foods or drinks by being mixed with various foods or drinks, regardless of the form such as liquid, paste, powder, or solid. Examples of the foods or drinks can include milk, soft drinks, powder beverages, fermented milk, lactic acid bacteria beverages, acidic beverages, yogurt, cheese, bread, biscuits, crackers, pizza crusts, modified milk powder, liquid foods, foods for patients, nutritious foods, frozen foods, food compositions, processed foods, and other commercial foods. In the case where the “composition for suppressing exercise-induced hemolysis, using (containing) astaxanthin as an active ingredient” and the “composition for suppressing and/or improving exercise-induced hemolytic anemia, using (containing) astaxanthin as an active ingredient” in the present invention are in the form of acidic agents or foods or drinks, the pH thereof can be set to 2.0 to 6.0, preferably 3.0 to 5.0. Further, the intake of free astaxanthin is generally about 0.03 mg to 100 mg, preferably about 0.05 mg to 60 mg, orally per adult per day, and may be appropriately increased or decreased according to the physical constitution or the symptoms.

The “composition for suppressing exercise-induced hemolysis, using (containing) astaxanthin as an active ingredient”, the “composition for suppressing and/or improving exercise-induced hemolytic anemia, using (containing) astaxanthin as an active ingredient”, the “food or drink for suppressing exercise-induced hemolysis, using (containing) astaxanthin as an active ingredient”, and the “food or drink for suppressing and/or improving exercise-induced hemolytic anemia, using (containing) astaxanthin as an active ingredient” in the present invention can additionally contain other carotenoids, vitamins, peptides, minerals, organic acids, short-chain fatty acids, fatty acid esters, or organic bases, as long as the features of the present invention are not impaired, and they may further contain fragrances, sweeteners, acidulants, or colorants, and further various fats/oils, for the purpose of improving the taste or aesthetic appearance, as long as the features of the present invention are not impaired.

Further, in the case where the “composition for suppressing exercise-induced hemolysis, using (containing) astaxanthin as an active ingredient”, the “composition for suppressing and/or improving exercise-induced hemolytic anemia, using (containing) astaxanthin as an active ingredient”, the “food or drink for suppressing exercise-induced hemolysis, using (containing) astaxanthin as an active ingredient”, the “food or drink for suppressing and/or improving exercise-induced hemolytic anemia, using (containing) astaxanthin as an active ingredient”, the “exercise-induced hemolysis suppressant using (containing) astaxanthin as an active ingredient”, and the “exercise-induced hemolytic anemia suppressing and/or improving agent using (containing) astaxanthin as an active ingredient” in the present invention contain water, the water is not specifically limited as long as it is used as foods, pharmaceutical products, and cosmetics. For example, purified water, pure water, deionized water, alkali ion water, deep water, wave water, natural water, or the like can be used.

Further, the “composition for suppressing exercise-induced hemolysis, using (containing) astaxanthin as an active ingredient”, the “composition for suppressing and/or improving exercise-induced hemolytic anemia, using (containing) astaxanthin as an active ingredient”, the “food or drink for suppressing exercise-induced hemolysis, containing astaxanthin as an active ingredient”, the “food or drink for suppressing and/or improving exercise-induced hemolytic anemia, using (containing) astaxanthin as an active ingredient”, the “exercise-induced hemolysis suppressant using (containing) astaxanthin as an active ingredient”, and the “exercise-induced hemolytic anemia suppressing and/or improving agent using (containing) astaxanthin as an active ingredient” in the present invention may contain any substance, as long as the features of the present invention are not impaired.

### EXAMPLES

Hereinafter, the composition for suppressing and/or improving exercise-induced hemolytic anemia, using (containing) astaxanthin as an active ingredient according to the present invention will be described with reference to Examples. The technical scope of the present invention is not limited to these Examples.

### Example 1

### Confirmation Test for Suppression of Exercise-Induced Hemolysis Using Astaxanthin

A confirmation test to confirm whether or not exercise-induced hemolysis that causes exercise-induced hemolytic anemia can be suppressed by orally taking a test food containing astaxanthin by a subject was conducted as follows.

### [1-1] Test Foods and Subjects

Two types of test foods, a “capsules for astaxanthin group” containing a Haematococcus algae-derived pigment and a “capsules for control group” not containing the Haematococcus algae-derived pigment were prepared. Further, twenty-eight men's medium- and long-distance runners aged from 18 to 22 belonging to the athletic club of Tokai University as subjects were divided into two groups. Thirteen subjects were designated as the “astaxanthin group”, and fifteen subjects were designated as the “control group”. Table 1 below shows the names (raw material names) of the contents in the test foods, the “capsules for astaxanthin group” and the “capsules for control group”, and the amounts to be mixed, the daily intakes, and specifications.

TABLE 1

| | | Capsules for | Capsules for |
|---|---|---|---|
| | | astaxanthin group | control group |
| Contents | Haematococcus algae | 120 mg | 0 mg |
| | pigment | | |
| | Olive oil | 80 mg | 180 mg |
| | Caramel coloring | 0 mg | 20 mg |
| Film | Processed starch | 110 mg | 110 mg |
| | Gelling agent | | |
| | (thickening | | |
| | polysaccharide) | | |
| | Glycerin | | |

| | | |
|---|---|---|
| Capacity per a capsule | 310 mg | 310 mg |
| Astaxanthin content per a capsule | 6 mg | 0 mg |
| Daily astaxanthin intake | 12 mg/2 capsules | 0 mg/2 capsules |

| | |
|---|---|
| Shape/packaging | Black, oval-type soft capsule/ |
| | aluminum pouch packaging |
| Manufacturing date | June 2018 |

### [1-2] Test Method

The exercise-induced hemolytic anemia is anemia that is mainly caused by physical destruction of erythrocytes (hemolysis) due to the impact given to the soles and develops when the number of erythrocytes (red blood cells) destroyed (number of hemolysis) exceeds the number of erythrocytes newly created by hematopoiesis (number of hematopoiesis), as described above. Accordingly, this test was conducted by, after acclimatization of subjects in advance by conducting running training in the highlands where exercise-induced hemolysis is less likely to be observed in order to cause the number of hematopoiesis to exceed the number of hemolysis, conducting running training in flatlands where exercise-induced hemolysis is likely to be observed in order to cause the number of hemolysis to exceed the number of hematopoiesis and observing the rate of change in the number of erythrocytes, the rate of change in the hemoglobin content (amount of blood pigment; hemoglobin concentration), and the transition of serum haptoglobin concentration in the subjects in the “astaxanthin group” and the “control group” during the period of running training in the flatlands.

Specifically, running training in the highlands was first conducted for 4 weeks, setting the period of running training in the highlands as “acclimatization period (Weeks 0 to 4)”, then blood samples were collected from the subjects of the “astaxanthin group” and the “control group”, to measure the number of erythrocytes (red blood cells), the hemoglobin content (amount of blood pigment; hemoglobin concentration), and the serum haptoglobin concentration. Subsequently, running training in the flatlands was conducted for 4 weeks, setting the period of running training in the flatlands as “observation period (Weeks 4 to 8)”, and then blood samples were collected again from the subjects of the “astaxanthin group” and the “control group”, to measure the number of erythrocytes, the hemoglobin content (hemoglobin concentration), and the serum haptoglobin concentration. This test was conducted by observing the rate of change in the number of erythrocytes, the rate of change in the hemoglobin content (hemoglobin concentration), and the transition of the serum haptoglobin concentration from the start date to the end date of the observation period in each of the “astaxanthin group” and the “control group” thus measured. The “astaxanthin group” was allowed to take the “capsules for astaxanthin group” daily (the intake of astaxanthin was 12 mg/day in terms of free form), and the “control group” was allowed to take the “capsules for control group” daily.

### [1-3] Method for Measuring the Number of Erythrocytes (Red Blood Cells), Hemoglobin Content (Amount of Blood Pigment; Hemoglobin Concentration), and Serum Haptoglobin Concentration

The blood samples collected from the subjects of the “astaxanthin group” and the “control group” were treated with EDTA-2K to measure the number of erythrocytes using the sheath flow electrical resistance detection method. On one hand, the collected blood samples were treated with EDTA-2K to measure the hemoglobin content (hemoglobin concentration) using the Sodium Lauryl Sulfate-Hemoglobin (SLS-Hb) method. On the other hand, the collected blood samples were transferred to a tube containing EDTA-2Na and centrifuged to separate the plasma fraction, to measure the serum haptoglobin concentration using an ELISA kit for haptoglobin quantification (Quantikine Human Haptoglobin ELISA Kit (product code: DHAPGO), available from R&D Systems, Inc.).

### [1-4] Observation of Rate of Change in the Number of Erythrocytes (Red Blood Cells), Rate of Change in Hemoglobin Content (Amount of Blood Pigment; Hemoglobin Concentration), and Serum Haptoglobin Concentration

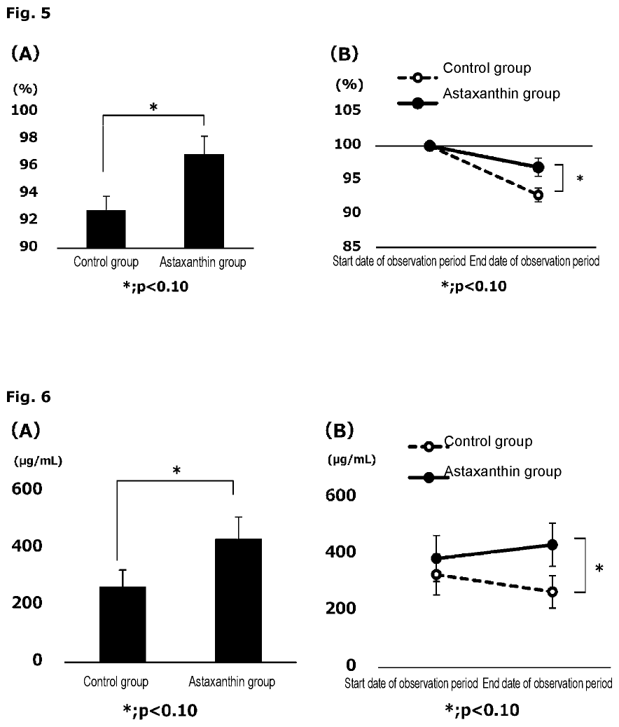

The number of erythrocytes and the hemoglobin content (hemoglobin concentration) on the start date of the observation period and the end date of the observation period in the “astaxanthin group” and the number of erythrocytes and the hemoglobin content (hemoglobin concentration) on the start date of the observation period and the end date of the observation period in the “control group” were each measured. Then, the measured values of the number of erythrocytes in the “astaxanthin group” and the “control group” on the start date of the observation period and the measured values of the hemoglobin content (hemoglobin concentration) were each taken as 100, to calculate the percentage of the measured values of the number of erythrocytes and the percentage of the measured values of the hemoglobin content (hemoglobin concentration) on the end date of the observation period. The percentages are respectively taken as the “rate of change in the number of erythrocytes during the observation period” and the “rate of change in the hemoglobin content (hemoglobin concentration) during the observation period”. Thereafter, the averages of the rate of change in the number of erythrocytes during the observation period and the averages of the rate of change in the hemoglobin content (hemoglobin concentration) in the “astaxanthin group” and the “control group” were compared with each other. The averages of the rate of change in the number of erythrocytes and the averages of the rate of change in the hemoglobin content (hemoglobin concentration) were compared between the “astaxanthin group” and the “control group” by conducting an unpaired t-test. The hypotheses that there was no difference in the averages of the rate of change in the number of erythrocytes between the “astaxanthin group” and the “control group”, and there was no difference in the averages of the rate of change in the hemoglobin content (hemoglobin concentration) between the “astaxanthin group” and the “control group” were rejected at p<0.1. Further, the averages of the serum haptoglobin concentration in the “astaxanthin group” on the start date of the observation period and the end date of the observation period measured and the averages of the serum haptoglobin concentration in the “control group” on the start date of the observation period and the end date of the observation period measured were each calculated and compared. FIG. 4 shows the rate of change in the number of erythrocytes and its transition in each of the “astaxanthin group” and the “control group” during the observation period. FIG. 5 shows the rate of change in the hemoglobin content (hemoglobin concentration) and its transition in each of the “astaxanthin group” and the “control group” during the observation period. FIG. 6 shows the average of measured values of the serum haptoglobin concentration on the start date of the observation period and the end date of the observation period in each of the “astaxanthin group” and the “control group”, and the transition of the average of measured values of the serum haptoglobin concentration during the observation period.

FIG. 4(A) is a graph showing the rate of change in the number of erythrocytes (red blood cells) in each of the “astaxanthin group” and the “control group” during the observation period, and FIG. 4(B) is a graph showing the transition of the rate of change in the number of erythrocytes from the start date of the observation period to the end date of the observation period in each of the “astaxanthin group” and the “control group”. As shown in FIG. 4(A) and FIG. 4(B), the value of the rate of change in the number of erythrocytes during the observation period in the “astaxanthin group” was high, as compared with the value of the rate of change in the number of erythrocytes during the observation period in the “control group”. Further, FIG. 5(A) is a graph showing the rate of change in the hemoglobin content (amount of blood pigment; hemoglobin concentration) during the observation period in each of the “astaxanthin group” and the “control group”, and FIG. 5(B) is a graph showing the transition of the rate of change in the hemoglobin content (hemoglobin concentration) from the start date of the observation period to the end date of the observation period in each of the “astaxanthin group” and the “control group”. The value of the rate of change in the hemoglobin content (hemoglobin concentration) during the observation period in the “astaxanthin group” was also high, as compared with the value of the rate of change in the hemoglobin content (hemoglobin concentration) during the observation period in the “control group”. From these facts, it was clarified that the hemolysis, that is, the reduction of the number of erythrocytes due to physical destruction of erythrocytes and the reduction of the hemoglobin content (hemoglobin concentration) were suppressed in the “astaxanthin group”.

Meanwhile, FIG. 6(A) is a graph showing the average of measured values of the serum haptoglobin concentration on the end date of the observation period in each of the “astaxanthin group” and the “control group”, and FIG. 6(B) is a graph showing the transition of the average of measured values of the serum haptoglobin concentration from the start date of the observation period to the end date of the observation period in each of the “astaxanthin group” and the “control group”. The degree of physical destruction of erythrocytes (red blood cells), that is, the degree of hemolysis can be known by measuring the serum haptoglobin concentration, and the reduction of the serum haptoglobin concentration indicates that hemolysis has occurred. Although there was almost no difference between the value of the serum haptoglobin concentration on the start date of the observation period in the “astaxanthin group” and the value of the serum haptoglobin concentration in the “control group”, as shown in FIG. 6(B), the value of the serum haptoglobin concentration on the end date of the observation period in the “astaxanthin group” was extremely high as compared with the value of the serum haptoglobin concentration on the end date of the observation period in the “control group”, as shown in FIG. 6(A) and FIG. 6(B). This revealed that hemolysis, that is, physical destruction of erythrocytes was suppressed in the “astaxanthin group”.

The aforementioned results revealed that the test food containing astaxanthin has an effect of suppressing hemolysis, that is, physical destruction of erythrocytes (red blood cells), an effect of suppressing the reduction of the number of erythrocytes, and the effect of suppressing the reduction of the hemoglobin content (amount of blood pigment; hemoglobin concentration). Therefore, it was indicated that a food or drink or an agent containing astaxanthin suppressed exercise-induced hemolysis and further suppressed and improved exercise-induced hemolytic anemia.

### Example 2

### Confirmation Test for Presence or Absence of Suppression of Hemolysis Due to Oxidative Damage in “Astaxanthin Group”

In order to confirm whether or not hemolysis due to oxidative damage is suppressed in the “astaxanthin group” of Example 1 (whether or not oxidative damage to erythrocytes (red blood cells) is suppressed), the production rate of 8-OHdG and isoprostane contained in the urine of the subjects of the “astaxanthin group” and the “control group” was investigated during the “test period” of Weeks 0 to 8 through the “acclimatization period (Weeks 0 to 4)” and the “observation period (Weeks 4 to 8)” of Example 1. The period of the confirmation test was set to the “test period” of Weeks 0 to 8 through the “acclimatization period (Weeks 0 to 4)” and the “observation period (Weeks 4 to 8)” because there was no causal relationship of whether or not hemolysis occurred due to oxidative damage with whether running training was performed in the highlands or flatlands.

Specifically, this test was performed as follows. Urine was collected from the subjects of the “astaxanthin group” and the “control group” at Week 0 (start date of test food intake), Week 4, and Week 8 (last day of test food intake), and the production rate of 8-OHdG and the production rate of isoprostane were measured. Thereafter, their averages were calculated, and then the transitions of the averages of the production rate of 8-OHdG and the production rate of isoprostane from Week 0 to Week 8 in each of the “astaxanthin group” and the “control group” thus calculated were observed.

### [2-1] Method for Measuring Production Rate of 8-OHdG and Production Rate of Isoprostane

The 8-OHdG concentration in the urine collected from the subjects of the “astaxanthin group” and the “control group” was measured using a New 8-OHdG Check ELISA (ELISA kit for 8-OHdG (product code: KOG-2005/E), available from NIKKEN SEIL Co., Ltd.) by an ELISA method. The production rate of 8-OHdG per body weight and time was calculated by the formula shown below.

Production rate of 8-OHdG (ng/kg/hr)={8-OHdG concentration in urine (ng/mL)×volume of urine collected (mL)}/{elapsed time since the last urination (h)×body weight of subject (kg)}

The isoprostane concentration in the urine collected from the subjects of the “astaxanthin group” and the “control group” was measured using a urinary isoprostane measurement ELISA kit (8-iso-PGF2a ELISA kit (product code: ADI-900-010), available from Enzo Life Sciences) by an ELISA method. The production rate of isoprostane per body weight and time was calculated by the formula shown below.

Production rate of isoprostane (ng/kg/hr)={isoprostane concentration in urine (ng/mL)×volume of urine collected (mL)}/{elapsed time since the last urination (h)×body weight of subject (kg)}

### [2-2] Statistical Method of Averages of Production Rate of 8-OHdG and Production Rate of Isoprostane and Observation

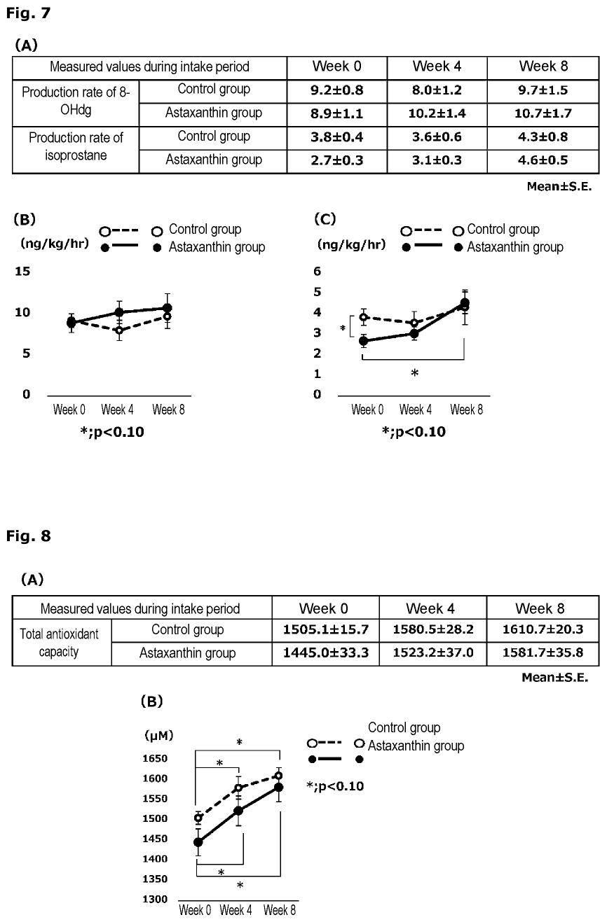

The averages of the production rate of 8-OHdG and the production rate of isoprostane during the test period (from Week 0 to Week 8) were compared between the “astaxanthin group” and the “control group” by conducting an unpaired t-test, and a hypothesis that there was no difference in the averages between the “astaxanthin group” and the “control group” was rejected at p<0.1. Further, the averages of the production rate of 8-OHdG and the production rate of isoprostane were compared between Week 0 and Week 4 or between Week 0 and Week 8 in each of the “astaxanthin group” and the “control group” by conducting the Dunnett's test, and a hypothesis that there was no difference in the averages between Week 0 and Week 4 or in the averages between Week 0 and Week 8 was rejected at p<0.1. FIG. 7 shows the averages of measured values of the production rates of 8-OHdG and isoprostane at Week 0 (start date of test food intake), Week 4, and Week 8 (last day of test food intake), and the transitions of the averages of measured values of the production rates of 8-OHdG and isoprostane during the test period, in each of the “astaxanthin group” and the “control group”.

FIG. 7(A) is a table showing the averages of measured values of the production rates of 8-OHdG and isoprostane at Week 0, Week 4, and Week 8 in each of the “astaxanthin group” and the “control group”, FIG. 7(B) is a graph showing the transition of the average of measured values of the production rate of 8-OHdG from Week 0 to Week 8, that is, during the test period in each of the “astaxanthin group” and the “control group”, and FIG. 7(C) is a graph showing the transition of the average of measured values of the production rate of isoprostane from Week 0 to Week 8, that is, during the test period in each of the “astaxanthin group” and the “control group”. Urinary 8-OHdG is a biomarker reflecting the oxidative damage to DNA due to oxidative stress such as active oxygen. The magnitude of oxidative stress and the level of oxidative damage to DNA can be evaluated by measuring the production rate of 8-OHdG. Further, urinary isoprostane is a prostaglandin-like compound formed by oxidation of phospholipids by free radicals, and lipid oxidation in vivo can be evaluated by measuring the production rate of isoprostane. As shown in FIG. 7(A), FIG. 7(B), and FIG. 7(C), there was no significant difference in the average of measured values of the production rate of 8-OHdG during the test period between the “astaxanthin group” and the “control group”, whereas the average of measured values of the production rate of isoprostane at Week 0 in the “astaxanthin group” was low as compared with that in the “control group”, but there was no difference observed in that at Week 4 and Week 8 between the “control group” and the “astaxanthin group”. This revealed that hemolysis due to oxidative damage was not suppressed in the “astaxanthin group” (oxidative damage to erythrocytes (red blood cells) was not suppressed). As a result of comparison between before and after taking the test foods, no difference was observed in the “control group”, whereas the average of the “astaxanthin group” increased after intake, and thus there was a difference observed as compared with before intake.

The aforementioned results revealed that the effect of suppressing hemolysis, that is, physical destruction of erythrocytes (red blood cells) exerted by the test food containing astaxanthin in Example 1 and the effect of suppressing the reduction of the number of erythrocytes were not associated with the effect of suppressing hemolysis due to oxidative damage, that is, oxidative damage to erythrocytes, which is supposed to be due to astaxanthin.

### Example 3

### Confirmation Test for Antioxidant Capacity in “Astaxanthin Group”

In order to confirm the antioxidant capacity in the “astaxanthin group” of Example 1, the concentrations of water-soluble antioxidants contained in the serum of the subjects of the “astaxanthin group” and the “control group” were measured during the “test period” of Weeks 0 to 8 through the “acclimatization period (Weeks 0 to 4)” and the “observation period (Weeks 4 to 8)” of Example 1. The period of the confirmation test was set to the “test period” of Weeks 0 to 8 through the “acclimatization period (Weeks 0 to 4)” and the “observation period (Weeks 4 to 8)” because there was no causal relationship of the antioxidant capacity with whether running training was performed in the highlands or flatlands.

Specifically, this test was performed as follows. The blood samples were collected from the subjects of the “astaxanthin group” and the “control group” at Week 0 (start date of test food intake), Week 4, and Week 8 (last day of test food intake), to measure the concentrations of water-soluble antioxidants in serum and measure the total antioxidant capacity. Thereafter, the average was calculated, and the transition of the average of the total antioxidant capacity from Week 0 to Week 8 in each of the “astaxanthin group” and the “control group” thus calculated was observed.

### [3-1] Method for Measuring Total Antioxidant Capacity

The blood samples collected from the subjects of the “astaxanthin group” and the “control group” were centrifuged to separate the serum fraction, to measure the concentrations of water-soluble antioxidants in serum using an antioxidant capacity measurement kit (Randox Total Antioxidant Status (product code: NX2332), available from RANDOX REAGENTS) by colorimetry and to measure the total antioxidant capacity.

### [3-2] Statistical Method of Average of Total Antioxidant Capacity and Observation

The averages of the total antioxidant capacity during the test period (from Week 0 to Week 8) were compared between the “astaxanthin group” and the “control group” by conducting an unpaired t-test, and a hypothesis that there was no difference in the averages between the “astaxanthin group” and the “control group” was rejected at p<0.1. Further, the averages of the total antioxidant capacity were compared between Week 0 and Week 4 or between Week 0 and Week 8 in each of the “astaxanthin group” and the “control group” by conducting the Dunnett's test, and a hypothesis that there was no difference in the averages between Week 0 and Week 4 or in the averages between Week 0 and Week 8 was rejected at p<0.1. FIG. 8 shows the averages of measured values of the total antioxidant capacity at Week 0 (start date of test food intake), Week 4, and Week 8 (last day of test food intake) in each of the “astaxanthin group” and the “control group”, and the transition of the average of measured values of the total antioxidant capacity during the test period.

FIG. 8(A) is a table showing the average of measured values of the total antioxidant capacity at Week 0, Week 4, and Week 8 in each of the “astaxanthin group” and the “control group”, and FIG. 8(B) is a graph showing the transition of the average of measured values of the total antioxidant capacity from Week 0 to Week 8, that is, during the test period in each of the “astaxanthin group” and the “control group”. The total antioxidant capacity (Serum Total Antioxidant Status) against oxidative stress can be known by detecting the water-soluble antioxidants in the serum. As shown in FIGS. 8(A) and (B), there was no significant difference in the average of measured values of the total antioxidant capacity during the test period between the “astaxanthin group” and the “control group”. This revealed that there was no difference in antioxidant capacity between the “astaxanthin group” and the “control group”.

The aforementioned results revealed that the effect of suppressing hemolysis, that is, physical destruction of erythrocytes (red blood cells) exerted by the test food containing astaxanthin in Example 1 and the effect of suppressing the reduction of the number of erythrocytes were not associated with the antioxidant capacity of astaxanthin.

1. A composition for suppressing exercise-induced hemolysis, using astaxanthin as an active ingredient.

2. A composition for suppressing and/or improving exercise-induced hemolytic anemia, using astaxanthin as an active ingredient.

3. The composition according to claim 1, wherein the composition is a food or drink composition or a pharmaceutical composition.

4. A food or drink for suppressing exercise-induced hemolysis or for suppressing and/or improving exercise-induced hemolytic anemia, using astaxanthin as an active ingredient.

5. (canceled)

6. The composition according to claim 2, wherein the composition is a food or drink composition or a pharmaceutical composition.
